(19) United States
(12) Patent Application Publication
(10) Pub. No.: US 2025/0235605 A1
(43) Pub. Date: Jul. 24, 2025

(54) AN ANAL IRRIGATION PROBE

(71) Applicant: Coloplast A/S, Humlebaek (DK)

(72) Inventor: Lars Olav Schertiger, Fredensborg (DK)

(21) Appl. No.: 18/853,450

(22) PCT Filed: Mar. 29, 2023
(86) PCT No.: PCT/DK2023/050069

§ 371 Date: Oct. 2, 2024

(30) Foreign Application Priority Data
Apr. 8, 2022 (DK) PA 2022 70193

(51) Int. Cl. A61M 3/02; A61M 25/10

(52) U.S. Cl. CPC A61M 3/0295; A61M 25/10185; A61M 2210/1067

(57) ABSTRACT

An anal probe is provided. The anal probe has an outer part comprising a balloon element and an inner part comprising a valve element. The valve element is configured to transition between closed and open position, when the pressure inside the inner part reaches a first threshold value. Thereby, the probe by itself can shift between inflating the balloon and irrigating the bowels. Furthermore, the outer part can be discarded following each use, whereas the inner part can be re-used.

The invention relates to an anal irrigation probe and an anal irrigation system including an anal irrigation probe.

### BRIEF DESCRIPTION OF THE DRAWING

The accompanying drawings are included to provide a further understanding of embodiments and are incorporated into and a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 illustrates an anal irrigation system as described herein.

FIGS. 2 to 7 illustrate an example of an anal probe as described herein. FIGS. 3, 5, 6 and 7 illustrate cross-sectional views, whereas FIGS. 2 and 4 illustrate side views. FIGS. 2 and 3 illustrate the probe in un-inflated state, FIGS. 4 and 5 illustrate the probe in inflated state. FIGS. 6 and 7 illustrate an outer part and an inner part separately.

FIGS. 8 and 9 illustrate further examples of an anal probe as described herein, both in cross-sectional views. In FIG. 8 only an inner part is illustrated, whereas in FIG. 9 an assembled probe is illustrated.

### DETAILED DESCRIPTION

Examples relate to an anal probe configured for being used in an anal irrigation system and for being inserted into the rectum of a user,
the anal probe comprising
   an outer probe part comprising
      a tip portion in a proximal end of the probe, the tip portion having at least one outlet opening for allowing irrigation liquid to flow out of the probe,
      an inflatable balloon at a middle portion of the outer probe part
      an interior lumen communicating with the at least one outlet opening and being configured for allowing irrigation liquid to pass through the lumen, the interior lumen further being configured for at least partly receiving an inner probe part
   an inner probe part comprising
      a valve element
      at least one inflation opening communicating with the inflatable balloon of the outer probe part
      at least one irrigation opening in a proximal portion of the inner probe part
wherein the valve element is a one-way valve element, which is configured for being closed, when a pressure distally of the valve element is below a threshold value and
wherein the valve element is configured for being open, when a pressure distally of the valve element exceeds the threshold value, and wherein the at least one irrigation opening is closed, when the valve element is in a closed state, and
wherein the outer probe part and the inner probe part comprises means for detachably attaching the inner probe part to the outer probe part, such that the two parts together constitutes an anal probe.

Examples relate to a method of using a probe as described above, wherein the user inserts the probe and allows it to shift between inflation and irrigation by itself.

Examples relate to an anal irrigation system comprising a container, a pump, tubing and an anal probe as described above.

Examples relate to a kit of parts comprising a single inner part of an anal probe as described above and a number of outer parts of an anal probe as described above.

When using an anal probe as described above, the anal probe will automatically shift between inflation of the balloon and discharge of irrigation liquid through the outlet openings. This provides an anal probe which is easy and simple to use. A related advantage is that the user does not have to figure out how to use a control unit, which in other systems are used to shift the system between inflating the balloon and irrigating the bowels. Furthermore, the anal probe will be safe to use, because the valve element and the first threshold value for transitioning between closed and open state will prevent the balloon from over-inflating. Thus, a probe as described will prevent or at least minimise the risk of the balloon bursting. Moreover, the user does not have to “prime” the probe prior to use. By priming is meant that the user empties the tubing from air entrapped in these tubing prior to use. This is done by pumping irrigation liquid into the tubing until the point, where the liquid reaches the probe. This is done to prevent the entrapped air from entering into the bowels, which may lead to painful cramps. In an anal probe as described herein, the priming step is not necessary, because any entrapped air in the tubing will enter into the balloon and assist in inflating the balloon, rather than enter into the bowels. The amount of air in the tubing will not be enough to induce a pressure distally of the valve element to reach the first threshold value, thus when all air has left the tubing and may have entered into the balloon, the pressure distally of the valve element will still be below the first threshold value. This means that the when the liquid front reaches the probe, the liquid will initially enter into the balloon and only in a later stage have induced a pressure high enough to exceed the first threshold value and thus cause the valve element to transition between a closed state and an open state.

By providing the probe in an inner part and an outer part, a cheap probe is obtained. This is because the outer part can be made very simple and this is configured to be used only once, whereas the inner part can be made more complex, because this is configured to be re-used.

In the following, whenever referring to a proximal end of end element described herein, the referral is to the end closest to the user of the system. Whenever referring to the distal end of an element, the referral is to the end opposite of that. This means that the insertable portion and in particular the outlet opening of the probe is in a proximal portion of the probe. The interior lumen of the probe extends in an axial direction. The direction transverse to the axial direction may be referred to as the transverse direction.

An irrigation system typically comprises a reservoir or container for irrigation liquid, an anal probe and tubing connecting those two. The system typically also include a pump for pumping the irrigation liquid into the intestines. The pump may be a manual pump or an electric pump. The pump may be configured for pumping air into the container to pressurise it, and thus allow liquid for being displaced from the container. The pump may be a liquid pump configured to directly displace the liquid from the container.

The anal probe comprises an insertable portion, which is configured for being inserted into the rectum or stoma of a user. Generally, the probe comprises a cylindrical outer body having a size of about 8-16 mm as the diameter such as 10 mm and a length of about 70-200 mm such as about 150 mm.

In a proximal portion of the insertable portion, the probe is provided with at least one outlet opening for letting irrigation liquid leave the probe and enter into the bowels of a user during use. The at least one outlet opening may be an open end of the insertable portion. Alternatively, the proximal end of the insertable portion may be closed and the outlet opening may be in the form of at least one, typically two transversely extending eyelet(s).

Another option is to provide the proximal end of the insertable portion with a number of outlet openings.

The distal portion of the probe may comprise a connector for connecting a tube to the probe. The distal portion may also be configured for having a tube attached to the probe by a non-detachable connection, e.g. by welding or adhering.

The probe comprises an outer part and an inner part. The outer part is configured for being used only once, whereas the inner part is configured for being re-used. Thus, the outer part and the inner part are detachably connected. The outer part is configured for being in contact with the rectum during the irrigation procedure, whereas the inner part is configured for being protected inside the outer part and for not being in contact with the rectum. The inner part includes the valve for shifting between inflation mode and irrigation mode, whereas the outer part includes the balloon and the outlet opening(s).

The outer part of the probe comprises a tube part having an interior lumen. A distal portion of the lumen is configured for receiving the inner part and include means for detachably attaching the inner part. A proximal portion of the lumen is configured for providing liquid flow to and through the outlet opening(s).

The inner part of the probe comprises a tube part having an inner lumen.

In examples, the valve element is a one-way valve element, which is configured for transition from a closed state to an open state, when a pressure distally of the valve element exceeds a first threshold value.

In examples the first threshold value is about 1.5 to 2.5 atm. In examples, the first threshold value is 2.0 atm. In examples the first threshold value is 2.5 atm.

The probe comprises retention means in the form of an inflatable balloon.

The inflatable balloon comprises a balloon element, where the material of the balloon element may be stretchable under normal use conditions—e.g. when subjected to pressures below 2.5 atm. If the balloon is stretchable, the stretchability of the balloon element is that it requires a strength of between about 0.1 MPa to about 10.0 20 MPa to extend the balloon to twice its length—preferably between about 0.2 MPa to about 1.5 MPa. In other words, when the strain has reached 100% of the initial length, then the tension in the material is between about 0.1 MPa and about 10.0 MPa—preferably between about 0.2 MPa and about 1.5 MPa.

Examples relate to the balloon having a first recommended minimum inflation pressure and a second recommended maximum inflation pressure. The first recommended minimum inflation pressure may be in the range 1.3 to 1.5 atm. The second recommended maximum inflation pressure may be in the range 2.7 to 3.0. The first threshold value for the pressure distally of the valve element may be between the first recommended minimum inflation pressure and the second recommended maximum inflation pressure of the balloon. This is one way of ensuring that the valve does not open before the balloon is sufficiently inflated and also does open before the balloon becomes over-inflated. In other words, this is one way of providing a probe which is easy and safe to use in the sense that the user does not have to worry about correctly inflating the balloon because this is automatically ensured by the configuration of the balloon and the valve.

Examples relate to an anal irrigation system where the balloon has a recommended minimum inflation volume, which exceeds the total volume of the tubing and the interior lumen of the outer probe part. This is one way of ensuring that the total volume of air present in the system prior to inflation of the balloon can be contained within the balloon without over-inflating the balloon. In other words, the automatic priming of the system happens by the air present in the system to begin with being pushed from the tubing and the interior lumen of the probe into the balloon by means of water from the container being pumped into the tubing and, later, into the probe. With the balloon being able to accommodate all the air initially present in the tubing and the probe, there will be no air left in the system, except in the balloon, once the valve opens. And therefore, there will be no risk of air being released into the bowels of the user. The recommended minimum inflation volume of the balloon may be in the range 35 ml to 75 ml.

The probe is provided with an inflatable balloon, which may be in the form of a stretchable balloon element. A stretchable balloon-element may be made of PU (Poly-Urethane), latex or PVC (Polyvinyl-chloride) although other materials having similar properties may be used. Other types of material may be silicone elastomers (e.g. LSR, HTV, RTV, Addition Cure or Condensation Cure), natural rubber latex, isoprene rubber, chloroprene rubber (e.g. Neoprene®), PU (poly-urethane, e.g. TPU or cross-linked), styrenic elastomers (TPE-S, e.g. Kraton®, Septon®, Sibstar® reactor materials or compounds) and poly-ether block amide (TPE-A), e.g. Pebax®).

Alternatively, the material is not stretchable in the normal use-condition that is when subjected to internal pressures below 2.5 atm. This means that the balloon element may be a moulded element made of a material which is not stretchable. The same kind of materials as mentioned above may be used for these balloons, with the addition that they need not be stretchable. Furthermore, materials like PET (polyethylene teraphthalate), PE (polyethylene), PP (polypropylene), Nylon, styrene and TPU may be used.

The tube part of the outer part of the probe may be made of recycled plastic materials and in particular, plastic material that is able to be recycled. PP is one example of a material, which can be recycled, another example is PE. Both of these examples are suitable for manufacturing the tube part of the outer part. The outer probe part is configured for being used only once, and then discarded—therefore, the material does not have fulfil any requirements as to being able to be cleaned and so forth.

The inner part of the probe may be configured for being re-used and may be re-used up to three months or even up to 6 months. With an irrigation procedure typically done at least every second day, this puts a certain demand on the inner part of the probe. In examples, the tube part of the inner part of the probe may be made of well-known plastic materials, such as acrylonitrile butadiene styrene, PP or PE.

Examples relate to the probe comprising a first sealing element positioned proximally of the inflation opening and distally of the valve element.

Examples relate to the probe comprising a second sealing element positioned distally of the inflation opening.

The sealing elements may be made of rubber or TPE.

Examples relate to the tip portion being atraumatic rounded, such as half-spherical in cross-section. An atraumatic rounded tip portion may be an advantage because the probe is configured to be inserted into the rectum. During this insertion, the probe may come into contact with soft tissue, thus preventing any unintentional impact on this soft tissue, an atraumatic tip portion may be helpful.

In an example the probe comprises two outlet openings, where each of the openings are positioned in a sidewall such that they face transversely to the longitudinal direction of the probe. In an example the probe has an open-ended tip portion. For example, the probe may have an outer sidewall, which extends from a distal end to a proximal end, and which has a through-going opening (the outlet opening) in a proximal end thereof.

In an example the probe comprises a number of outlet openings, such as 8, 10 or 12 openings, which are positioned in a rounded tip of the probe and facing generally in the longitudinal direction. By facing generally in the longitudinal or axial direction is meant that a centre of each outlet opening defines an angle with respect to the axial direction, which is less than 45 degrees, such as less than 30 degrees. A number of outlet openings such as mentioned above may provide for a well-distributed flow out of the probe.

In examples, the inner part of the probe comprises a number of inflation openings, such as 2, 3, 4, 5, 6, 8, 10 or 12. A number of inflation openings may provide for a fast and even inflation of the balloon.

In an example, the probe comprises a tube-connector in a distal end. Tube-connectors on probes provide the advantage of allowing a tube to be easily connected to the probe. This allows the tube to be re-used irrespectively of how the probe is re-used. Alternatively, the tube may be directly attached to the probe. Because the inner part is re-useable, the tube can be permanently attached to this part and be re-useable at the same interval as the inner part.

In examples, the probe is configured for providing liquid flow through an inner part of the probe from a distal end of the inner part, through an inner lumen of the inner part and leaving the inner part either through the inflation opening(s) or through the irrigation opening(s). This provides for flow either directly to the balloon or directly through the lumen of the probe without having to go through a complex system of channels.

Examples relate to the valve element being configured for transitioning from an open state to a closed state, when a pressure distally of the valve element falls below a second threshold value. The second threshold value may in examples be 1.5 atm. The second threshold value may in examples be 1.0 atm.

To prevent the valve element from continuously transitioning between open state and closed state, the valve element may have a gap between the first threshold value, that forces the valve element to open and the second threshold value, that will close the valve element.

Examples relate to the valve being an umbrella valve. In those examples, the valve element may be made of silicone and rubber-materials.

Examples relate to the valve being a ball valve.

Examples relate to the means for detachably attaching the outer part to the inner part comprising threads. The means for detachably attaching the outer part to the inner part may comprise a bayonet coupling. Alternatively, the means may comprise snap-fitting.

Examples relate to the inner part of the probe comprising a protective wall protecting the valve from being “polluted” by faecal matter. The protective wall will in that case be positioned proximally of the valve, such that the valve is protected by faecal matter or liquid entering into the probe through the outlet openings and further travelling through the inner lumen of the outer part in a distal direction. In examples, the protective wall comprises a mesh material, e.g. a porous or microporous material such as Tyvek®. In examples, the protective wall comprises apertures.

Examples relate to the interior lumen of the outer part being one single lumen.

Examples relate to the interior lumen of the outer part being divided into a proximal lumen and a distal lumen divided by a transversely extending partition wall. A transversely extending partition wall will be positioned in a portion of the outer part, which is proximal to the inner part, when the probe is assembled. Such a transversely extending partition wall may assist in protecting a valve element, when this valve element is positioned in the proximal end of the inner part. This means that the valve is protected by faecal matter or liquid entering into the probe through the outlet openings and further travelling through the inner lumen of the outer part in a distal direction. The partition wall may comprise a mesh material, e.g. a porous or microporous material such as Tyvek®. In examples, the partition wall comprises apertures.

### DETAILED DESCRIPTION OF THE DRAWING

Initially, it shall be noted that the figures are schematic illustrations intended only to address the principles and functions of the anal probe described herein and are not to be considered limiting to the scope of the attached claims. Furthermore, the figures and particularly the individually illustrated elements are not necessarily to scale, neither individually nor in relation to each other.

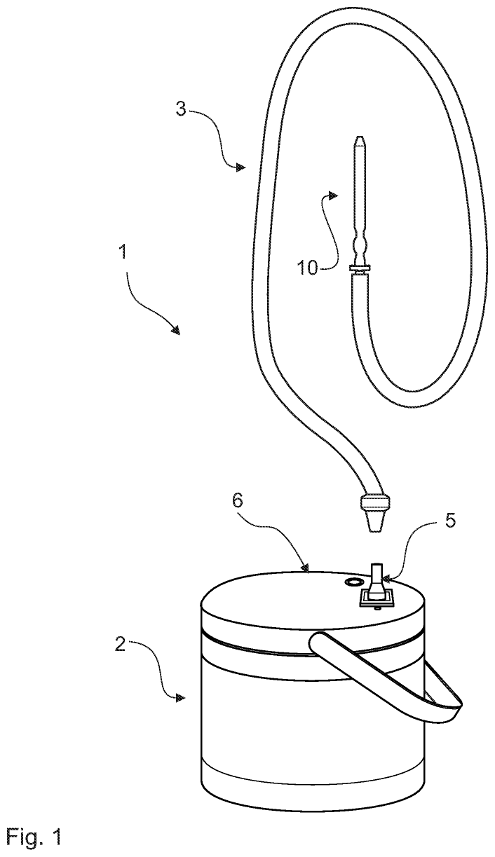

FIG. 1 illustrates an anal irrigation system 1. The system comprises a container for irrigation liquid 2, a tube 3 connected to an anal probe 10. A connector 5 connects the container 2 to the tube 3. In the example of the system illustrated in FIG. 1, the connector 5 is positioned in the lid 6 of the container. The container 2 may have other features, such as a handle, a scale, and so forth. The description here is mostly related to the anal probe 10, which will be described in more detail below.

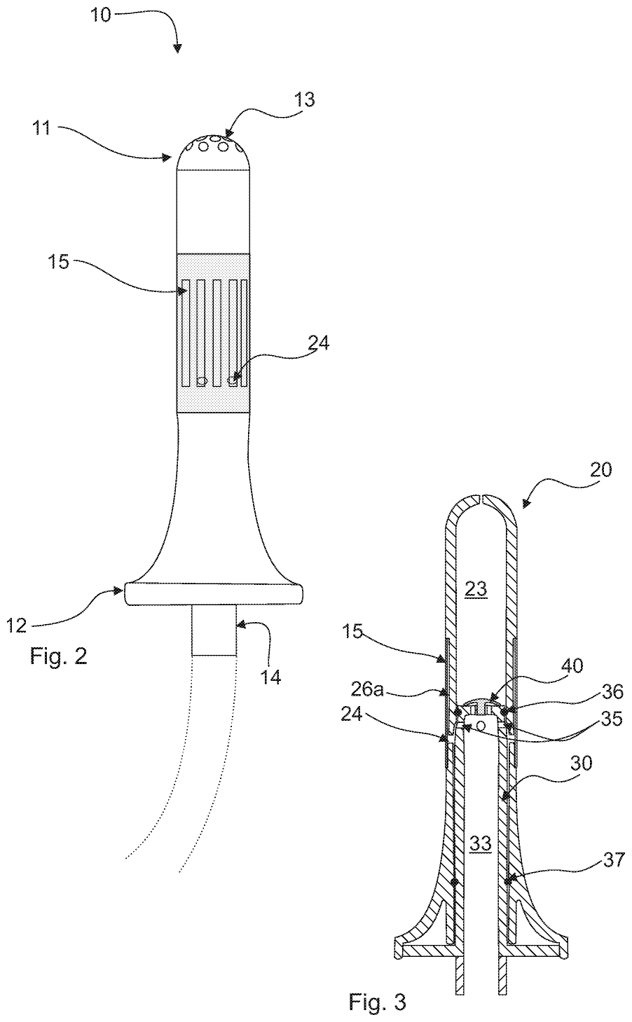

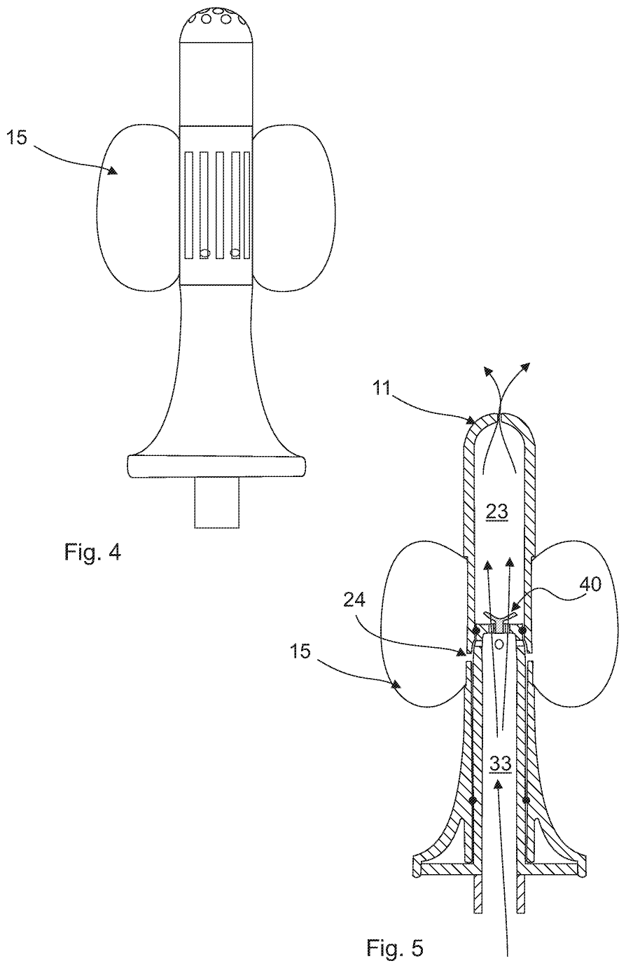

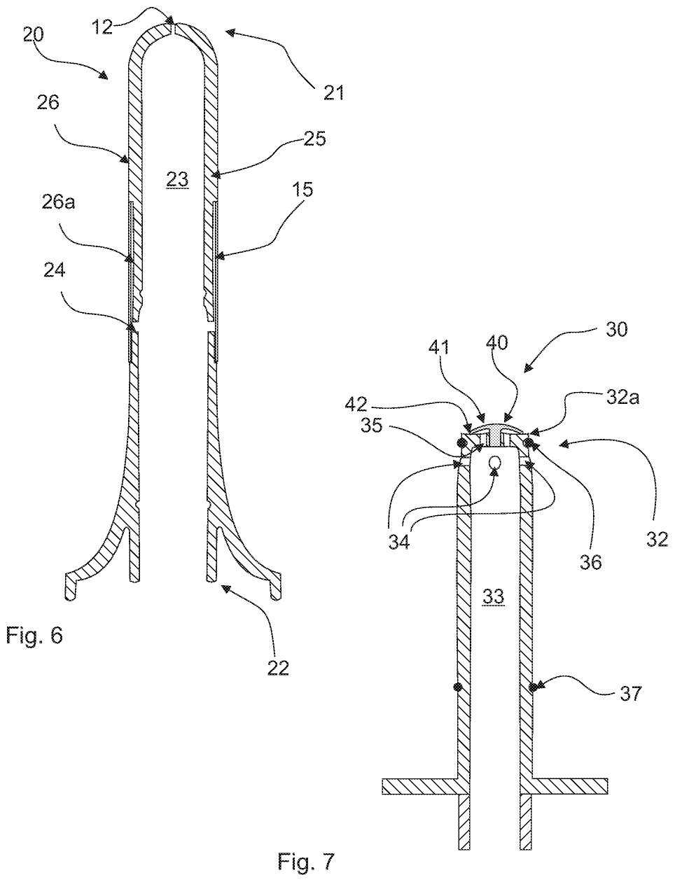

FIGS. 2-7 illustrates an anal probe 10 as described herein. In FIGS. 2 and 4, the probe is shown in a view showing the outer surface of the probe and FIGS. 3 and 5 illustrate cross-sectional views of the probe. In FIGS. 2 and 3 the balloon is deflated whereas it is illustrated as being inflated in FIGS. 4 and 5. FIGS. 6 and 7 illustrate cross-sectional views of an outer part and an inner part of the probe, respectively.

FIGS. 2 and 3 illustrates a probe comprising a proximal insertion end 11 and a distal end 12. The proximal end comprises a number of outlet openings 13. The illustrated probe is shown as comprising a number of outlet openings 13, 7 is shown in the side view of FIG. 2, but the probe may include 12 outlet openings or more. At the distal end 12 the probe comprises a connector 14 for connecting the probe to a tube; the tube is illustrated in dashed lines in FIGS. 2. The probe further comprises a balloon 15 on an outer surface of the probe.

In FIG. 3, it can be seen that the probe 10 comprises an outer part 20 detachably connected to an inner part 30; in FIG. 3, the two parts are shown connected to each other. In FIGS. 6 and 7, the two parts are shown separately. The outer part and inner part and their cooperation will be explained with reference to FIGS. 2 to 7.

The outer part 20 comprises a proximal end 21, which is the end of the probe configured for being inserted into the rectum. The outer part comprises a distal end 22, and an inner lumen 23 extending from a distal end to a proximal end. The outer part comprises a balloon channel 24 extends from an inner lumen 23 of the outer part through the wall 25 of the outer part to an outer surface 26 at a balloon-receiving portion thereof 26a, which is configured for having the balloon 15 attached to it. The balloon-receiving portion 26a of the surface is countersunk with respect to the remaining surface, such that when the balloon 15 is fitted in this portion, it does not protrude outside of the surface 26 (see FIG. 6).

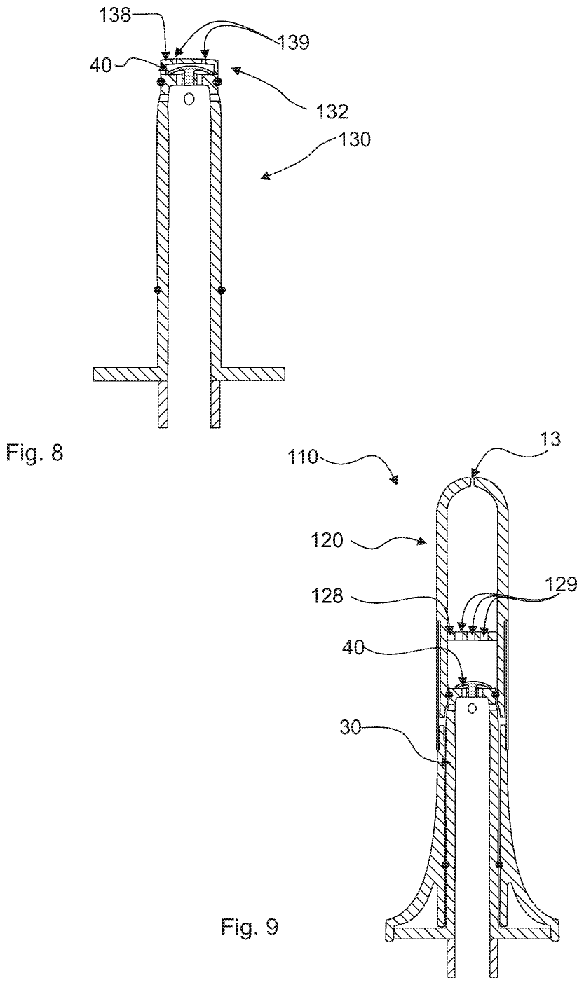

The inner part 30 has an inner lumen 33 extending from a distal end 31 to a proximal portion 32. The inner part comprises a one-way valve 40 in the proximal portion. In the illustrated example of FIG. 3, the valve 40 is positioned at the proximal end 32a of the inner part, but it may also be positioned below or distally of a protective wall as illustrated in FIG. 8. The valve element comprises a valve part 41 and a valve seat 42. The valve seat is at the proximal end 32a of the inner part as best seen in FIG. 7. The inner part has inflation openings 34, which are configured to provide liquid flow from the inner part to the balloon channel(s) 24 in the outer part. The inner part further has irrigation openings 35 positioned under the valve 40, so that they are closed, when the valve is closed and open when the valve is open. In the illustrated example, the inner part has a first sealing 36 in a proximal portion and a second sealing 37 in a distal portion. The first sealing 36 is positioned proximally of the inflation openings 34, such that liquid is prevented from bypassing the inflation opening in a distal direction without opening the valve element 40. The second sealing 37 is positioned distally of the inflation openings 34 such that liquid coming out of the inflation openings is prevented from seeping out of the probe in an interface between the inner part and the outer part. The position of the sealings 36, 37 in relation to the inflation openings 34 and the balloon channel 24 in the outer part is best seen in FIG. 3. This positioning ensures that the liquid either enters into the balloon or exits the probe at the outlet openings.

In FIGS. 4 and 5, the probe is illustrated with the balloon inflated. In FIG. 5, which is the cross-sectional view, the valve element is open. The arrows illustrate how liquid enters into the interior lumen 33 of the inner part, through the valve 40 to the inner lumen 20 of the outer part and finally exits at a proximal end 11 of the probe.

FIG. 8 illustrate an example of an inner part 130 of a probe as described herein. In the example of FIG. 8, the inner part comprises a protective wall 138 in the proximal end 132. This wall protects the valve element 40, which is situated distally of the protective wall 138. The valve element 40 will be able to transition to an open state within the interior space created by the protective wall 138. The protective wall 138 is in this illustrate example provided with apertures 139, such as to provide a pathway for liquid flowing past the valve element, when the valve element is in an open state.

FIG. 9 illustrate an example of a probe 110 as described herein. This probe 110 has an inner part 30 as described in relation to FIGS. 2 to 7, but the outer part 120 differs in that it is provided with a partition wall 128 positioned proximally of the inner part. The partition wall provides protection for the inner part 30 and in particular the valve element 40 for polluted liquid entering into the probe from the bowels through the outlet openings 113. The partition wall 128 is provided with apertures 129 so as to allow liquid to flow proximally through the partition wall 128 and out of the probe through the outlet openings 113. The partition wall may also be provided as a mesh structure, in which case the apertures may be dispensed with.

Embodiments, and features of the various exemplary embodiments described in this application, may be combined with each other (“mixed and matched”), unless specifically noted otherwise.

1-28. (canceled)

29. An anal probe insertable into a rectum of a user and attachable to an anal irrigation system, the anal probe comprising:
an outer part formed from a wall extending from a distal end to a proximal end, the outer part comprising:
a lumen formed longitudinally within the outer part,
an outlet opening formed through the wall at the proximal end of the outer part,
an inflatable balloon secured to an exterior surface of the wall of the outer part,
a balloon channel formed through the wall and communicating between the lumen and the inflatable balloon; and
an inner part detachably inserted into the lumen of the outer part, the inner part comprising:
a central lumen extending from a tube connector at a distal end to a valve assembly disposed at a proximal end of the inner part,
an irrigation opening formed in the proximal end of the inner part,
a valve flap of the valve assembly disposed over the irrigation opening,
an inflation opening formed through a sidewall the inner part and communicating with the central lumen,
a proximal seal disposed between the valve flap and the inflation opening;
wherein, when the inner part is inserted into the outer part:
the inflation opening aligns with the balloon channel of the outer part to allow a first portion of the irrigation liquid to inflate the inflatable balloon;
the valve flap of the valve assembly is movable away from the irrigation opening to allow a second portion of the irrigation liquid to exit the central lumen and the irrigation opening, flow through the lumen of the outer part, and exit the outlet opening formed at the proximal end of the outer part; and
the valve flap of the valve assembly is movable to cover the irrigation opening and prevent the irrigation liquid from moving from the lumen of the outer part and entering the irrigation opening of the inner part.

30. The anal probe of claim 29, wherein the inner part further comprises a distal seal positioned distal of the inflation opening.

31. The anal probe of claim 29, wherein the outlet opening formed through the wall at the proximal end of the outer part is one of a plurality of outlet openings formed through the wall at the proximal end of the outer part.

32. The anal probe of claim 29, wherein the irrigation opening formed in the proximal end of the inner part is one of a plurality of irrigation openings formed in the proximal end of the inner part.

33. The anal probe of claim 29, wherein the inner part, when inserted into the outer part, is isolated from an exterior of the outer part and adapted to be reused and the outer part is disposable.

34. The anal probe of claim 29, wherein the valve flap is umbrella-shaped and configured to lay over the irrigation opening.

35. The anal probe of claim 29, wherein the inner part is detachably inserted into the lumen of the outer part and retained by a threaded connection between the inner part and the outer part.

36. The anal probe of claim 29, wherein the inflatable balloon has a minimum inflation pressure and a maximum inflation pressure, and the valve flap of the valve assembly is movable away from the irrigation opening at a threshold valve pressure between the minimum inflation pressure and the maximum inflation pressure of the inflatable balloon.

37. The anal probe of claim 36, wherein the threshold valve pressure is selected to be in a range from 1.3 atmospheres to 3 atmospheres.
